(19) United States
(12) Patent Application Publication
(10) Pub. No.: US 2026/0224449 A1
(43) Pub. Date: Aug. 6, 2026

(54) SKIN CARE METHOD AND DUAL LIQUID SKIN CARE SET

(71) Applicant: Wenyan Li, Irvine, CA (US)

(72) Inventor: Wenyan Li, Irvine, CA (US)

(21) Appl. No.: 19/630,946

(22) Filed: Mar. 27, 2026

(30) Foreign Application Priority Data
Feb. 11, 2026 (CN) 202610203338.X

(51) Int. Cl. A61K 8/04; A61K 8/81; A61Q 19/00

(52) U.S. Cl. CPC A61K 8/042; A61K 8/8147; A61Q 19/00; A61K 2800/48; A61K 2800/87; A61K 2800/882

(57) ABSTRACT

Provided herein is a skin care method. The skin care method includes: providing a first liquid skin care composition, the first liquid skin care composition being a flowable liquid system with high-flowability, the first liquid skin care composition including a skin conditioning ingredient; providing a second liquid skin care composition, the second liquid skin care composition being a semi-flowable gel system, the second liquid skin care composition including a pH adjusting ingredient; and contact-mixing the first liquid skin care composition and the second liquid skin care composition during a skin use process. After the first liquid skin care composition and the second liquid skin care composition are contacted on a skin surface, through physical structure reorganization, a system is instantaneously transformed from a liquid or semi-flowable state into a viscoelastic gel-like structure or a film-forming state.

### CROSS-REFERENCE TO RELATED APPLICATIONS

The application claims priority of Chinese patent application CN202610203338.X, filed on 2026 Feb. 11, disclosure of which is incorporated herein by reference in its entirety.

### TECHNICAL FIELD

The present disclosure relates to the technical field of cosmetics, and in particular, to a skin care method and a dual liquid skin care set.

### BACKGROUND

With the continuous improvement of skin care needs, various forms of products have appeared in a skin care product market, including lotions, creams, essences, gels, and the like. Among these, liquid essence products are widely welcomed by consumers due to a light texture and rapid absorption. In the prior art, a liquid essence product typically adopts a single formulation system, and is pre-mixed during a production process into a specific texture, such as a water-like texture, a milky texture, or a gel-like texture, by adding ingredients such as a thickener and a film former.

In a prefabricated gel or gel product in the prior art, a polymeric network thereof has been formed before leaving a factory, and an active ingredient is in a restricted distribution state for a long term, and diffusion and release thereof are restricted by a network structure, affecting efficacy performance. At the same time, a texture of a prefabricated product is fixed, and a user cannot adjust the texture according to personal skin type or seasonal changes. Although dual-tube or dual-chamber packaging products exist on a market, these products typically merely provide simple layer-by-layer superposition, and two phases do not undergo structure-level interaction, failing to achieve texture transformation in a true sense and personalization of use experience.

Therefore, a skin care solution that may adjust a texture according to needs during use while ensuring stability and efficacy performance of an active ingredient is needed.

### SUMMARY

An objective of the present disclosure is to provide a skin care method and a dual liquid skin care set, aiming to solve technical problems in the prior art that a skin care product has a fixed texture and cannot be adjusted according to needs, and that an active ingredient is incompatible with a thickening system leading to decreased stability.

To achieve the above objective, the present disclosure provides a skin care method. The skin care method includes: providing a first liquid skin care composition, the first liquid skin care composition being a flowable liquid system and including a skin conditioning ingredient; providing a second liquid skin care composition, the second liquid skin care composition being a semi-flowable gel system and including a potential of hydrogen (pH) adjusting ingredient; and contact-mixing the first liquid skin care composition and the second liquid skin care composition during a skin use process. After the first liquid skin care composition and the second liquid skin care composition are contacted on a skin surface, through physical structure reorganization, a system is instantaneously transformed from a liquid or semi-flowable state into a viscoelastic gel-like structure or a film-forming state.

The present disclosure further provides a dual liquid skin care set. The dual liquid skin care set includes a first container and a second container. The first container contains a first liquid skin care composition, the first liquid skin care composition being a flowable liquid system and including a skin conditioning ingredient. The second container contains a second liquid skin care composition, the second liquid skin care composition being a semi-flowable gel system and including a pH adjusting ingredient. After the first liquid skin care composition and the second liquid skin care composition are contact-mixed during a skin use process, through physical structure reorganization, a system is instantaneously transformed from a liquid or semi-flowable state into a viscoelastic gel-like structure or a film-forming state.

Beneficial effects of the present disclosure may be as follows.

In the present disclosure, by triggering physical structure reorganization of the first liquid skin care composition and the second liquid skin care composition during a skin use process, a gel or film structure may be instantaneously generated at a point of use. Unlike an existing prefabricated gel or gel product, a gel structure of the present disclosure may be generated during use, rather than being stored before a storage stage. A newly generated polymeric network may be more relaxed and more elastic, and may not require sacrificing a skin feel for long-term storage or requiring excessive crosslinking. Compared with a long-term storage structure, such an instantaneously generated structure may have a better skin feel and higher use comfort.

In the present disclosure, an active ingredient may no longer be fixed in a polymeric network. The active ingredient may first diffuse and spread in a low-resistance environment and then be confined by a newly generated network, achieving a sequence of release followed by structuration. This may be in sharp contrast to a situation in a prefabricated gel where an active ingredient is trapped in a network for a long term and release thereof is restricted, and may significantly improve diffusion efficiency and efficacy performance of the active ingredient.

A structural state of the present disclosure may be triggered by a use method, rather than being preset by a formulation. A user may freely adjust according to needs, usage preferences, and a skin state: using only a first liquid to obtain a water-like refreshing effect, using only a second liquid to obtain a gel repair effect, mixing a small amount to obtain an essence gel texture, and mixing a large amount to obtain a mask film effect.

A flexible continuous film layer may be formed in the present disclosure, rather than a rigid or instantaneously occlusive film layer. A film-forming process may be gradual: a soft gel may be formed at an initial stage, a glossiness may increase at a middle stage, and a stable film layer may be formed at a later stage. Such gradual film formation may not rely on a strong occlusive agent, but may rely on gradual densification of a network, and thus may be non-occlusive, have good breathability, and have excellent skin compatibility.

### BRIEF DESCRIPTION OF THE DRAWINGS

Implementations of the present disclosure will now be described, by way of embodiment, with reference to the attached figures. It should be understood, the drawings are shown for illustrative purpose only, for ordinary person skilled in the art, other drawings obtained from these drawings without paying creative labor by an ordinary person skilled in the art should be within scope of the present disclosure.

FIG. 1 is a photograph of a first liquid skin care composition (upper), a second liquid skin care composition (middle), and a mixture of the first liquid skin care composition and the second liquid skin care composition (lower) after being spread on artificial leather.

FIG. 2 is a photograph of mixtures of the first liquid skin care composition and the second liquid skin care composition at different ratios after being spread on artificial leather, wherein from left to right, ratios are 1:0.5, 1:0.8, and 1:1.

FIG. 3 is a schematic structural view of a dual liquid skin care set according to an embodiment of the present disclosure.

### DETAILED DESCRIPTION OF THE EMBODIMENTS

It will be appreciated that for simplicity and clarity of illustration, where appropriate, reference numerals have been repeated among the different figures to indicate corresponding or analogous elements. In addition, numerous specific details are set forth in order to provide a thorough understanding of the exemplary embodiments described herein. However, it will be understood by those of ordinary skill in the art that the exemplary embodiments described herein may be practiced without these specific details. In other instances, methods, procedures, and components have not been described in detail so as not to obscure the related relevant feature being described. Also, the description is not to be considered as limiting the scope of the exemplary embodiments described herein. The drawings are not necessarily to scale and the proportions of certain parts may be exaggerated to better illustrate details and features of the present disclosure.

The term “comprising” when utilized, means “including, but not necessarily limited to”; it specifically indicates open-ended inclusion or membership in the so-described combination, group, series, and the like. The disclosure is illustrated by way of example and not by way of limitation in the figures of the accompanying drawings in which like references indicate similar elements. It should be noted that references to “an” or “one” embodiment in this disclosure are not necessarily to the same embodiment, and such references can mean “at least one”. In addition, the terms “first” and “second” are used for descriptive purposes only and cannot be understood as indicating or implying relative importance or implying the number of indicated technical features. Thus, the features defined as “first” and “second” may explicitly or implicitly include one or more of the features. In the description of embodiments of the application, “a plurality of” means two or more, unless otherwise specifically defined.

It should be noted that if ingredient content ranges (such as 0.1%-2%, 1:10 to 10:1, etc.) are involved in the embodiments of the present disclosure, the endpoint values of the ranges and any numerical values therebetween are within the protection scope. For example, “0.1%-2%” includes 0.1%, 0.5%, 1.0%, 1.5%, 2.0% and any numerical values therebetween. The same principle applies if process parameter ranges such as temperature, time, pH value are involved.

Additionally, if descriptions such as “first”, “second” are involved in the embodiments of the present disclosure, such descriptions of “first”, “second” are only used to distinguish different compositions or ingredients, and cannot be understood as indicating or implying their relative importance, order of addition, or implicitly indicating the quantity of the indicated technical features. Therefore, features defined with “first” and “second” may explicitly or implicitly include at least one of the features. Additionally, if “and/or” appears throughout the text, its meaning includes three parallel schemes. Taking “A and/or B” as an example, it includes scheme A, or scheme B, or a scheme where both A and B are satisfied. Additionally, the technical solutions between various embodiments may be combined with each other, but must be based on the ability of a person of ordinary skill in the art to achieve. When the combination of technical solutions contradicts or cannot be achieved, such a combination of technical solutions should be considered non-existent and also not within the protection scope claimed by the present disclosure.

The present disclosure provides a skin care method and a dual liquid skin care set.

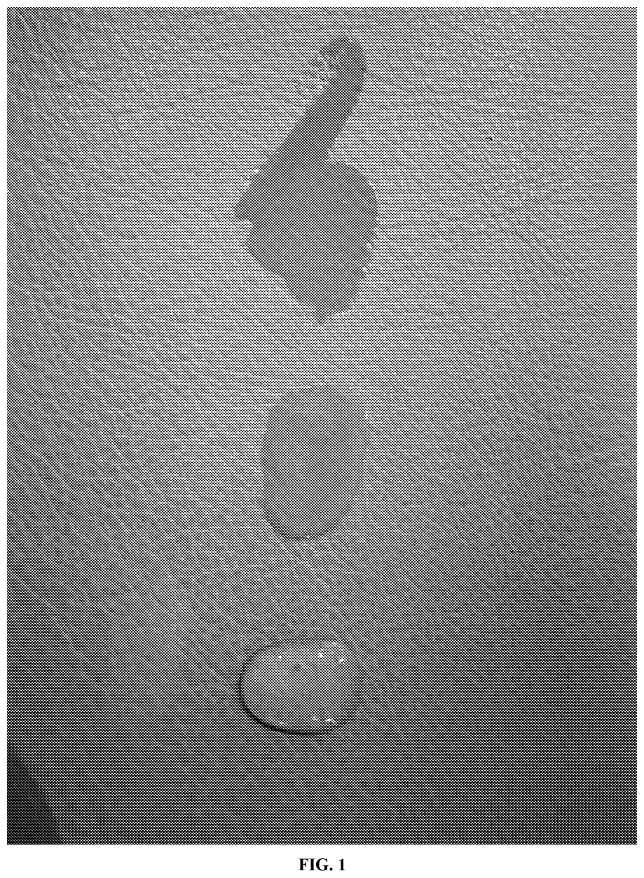

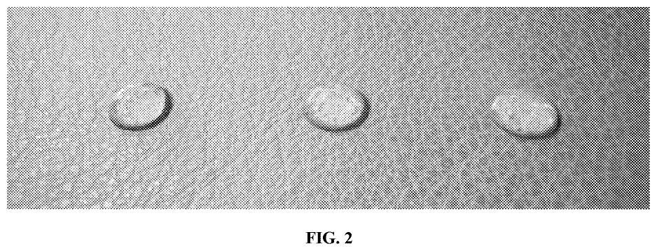

Referring to FIG. 1 and FIG. 2, the present embodiment provides a skin care method.

At step S100, a first liquid skin care composition 110 is provided. The first liquid skin care composition 110 is a flowable liquid system with high-flowability. The first liquid skin care composition 110 includes a skin conditioning ingredient.

In the present embodiment, the first liquid skin care composition 110 exhibits a watery liquid texture, has good flowability, and is easy to apply and absorb. The skin conditioning ingredient may be used to provide the skin with nutrition, antioxidant, whitening, or repair effects.

At step S200, a second liquid skin care composition 210 is provided. The second liquid skin care composition 210 is a semi-flowable gel system and includes a pH adjusting ingredient.

In the present embodiment, the second liquid skin care composition 210 exhibits a gel texture, has a certain viscosity but is still flowable. A polymeric thickener may form a colloid network structure under an action of the pH adjusting ingredient.

At step S300, the first liquid skin care composition 110 and the second liquid skin care composition 210 are contact-mixed during a skin use process.

In the present embodiment, a user may select a mixing method according to needs. The two compositions may be mixed first in a palm and then applied to skin, or applied separately and then mixed on a skin surface.

Referring to FIG. 1 and FIG. 2, after the first liquid skin care composition 110 and the second liquid skin care composition 210 are contacted on a skin surface, through physical structure reorganization, a system is instantaneously transformed from a liquid or semi-flowable state into a viscoelastic gel-like structure or a film-forming state. In the present embodiment, after the two liquid compositions are contact-mixed, an aqueous phase and a polyol ingredient in the first liquid skin care composition interact with a polymeric colloid network in the second liquid skin care composition, causing rearrangement of a colloid network structure, a viscosity of a system rapidly increases, forming a viscoelastic gel-like texture.

With the above technical solution, the present disclosure may achieve an instantaneous transformation of texture during use. A user may select to use a liquid essence alone or mix to use a gel-like product according to needs, flexibly meeting different skin care needs. At the same time, separating efficacy ingredients from a thickening system may avoid stability problems during long-term storage and ensure product efficacy.

It may be understood that, in some embodiments, a user may adjust a mixing ratio according to own skin type and seasonal changes, thereby obtaining different degrees of moisturizing and water-locking effects. For example, in summer or for oily skin, an amount of the second liquid skin care composition may be reduced to obtain a refreshing texture; in winter or for dry skin, an amount of the second liquid skin care composition may be increased to obtain a more abundant moisturizing effect.

In the present embodiment, the polymeric thickener includes at least one of: an acrylic polymer, and xanthan gum, or a combination thereof. An acrylic polymer may fully swell under the action of a pH adjusting agent to form a high-viscosity colloid network; xanthan gum, as a natural polysaccharide thickener, has good thickening and stabilizing effects.

Further, the acrylic polymer includes at least one of: carbomer, and crosslinked polyacrylic acid, or a combination thereof, and a content is 0.1%-2% of a total weight of the second liquid skin care composition.

In the present embodiment, carbomer is preferably used as a main thickener. Carbomer is a type of crosslinked polyacrylic acid polymer, and exists in a tight coil state under acidic conditions. After an alkaline pH adjusting agent is added, carboxyl groups on a molecular chain ionize, and electrostatic repulsion causes molecular chain extension, forming a high-viscosity colloid.

When a carbomer content is 0.1%, a light gel texture may be formed; when a content is 0.6%-1.0%, a gel with a moderate viscosity may be formed; when a content is 1.5%-2%, a high-viscosity gel may be formed. By adjusting a carbomer content, a base viscosity of the second liquid skin care composition may be controlled, thereby affecting a final texture after mixing with the first liquid skin care composition.

In the present embodiment, by selecting an appropriate thickener type and amount, an effect that the second liquid skin care composition maintains a semi-flowable state during storage and may be rapidly thickened when mixed is achieved. Compared with traditional thickeners, a carbomer system has advantages such as high transparency, refreshing skin feel, and high thickening efficiency, and may achieve an ideal thickening effect at a low usage amount.

It may be understood that, in some embodiments, other acrylic polymers may also be used, such as sodium polyacrylate, hydroxyethyl acrylate, or sodium acrylate copolymer, etc. These polymers may also achieve a thickening effect when a pH changes. Carbomer and xanthan gum may also be compounded and used, utilizing a synergistic thickening effect of the carbomer and the xanthan gum to obtain a more stable gel structure.

In the present embodiment, the pH adjusting ingredient includes an alkaline substance, used to neutralize acidic groups in a polymeric thickener, causing swelling and thickening.

The alkaline substance is selected from the group consisting of: triethanolamine, sodium hydroxide, potassium hydroxide, and tromethamine. In the present embodiment, triethanolamine is preferably used as a pH adjusting agent. Triethanolamine is a mild organic base, capable of effectively neutralizing carbomer, while having low skin irritation and high safety. Sodium hydroxide and potassium hydroxide are inorganic bases, have strong neutralization ability, and are used in small amounts, but precise amount control is required to avoid over-neutralization. Tromethamine (tris(hydroxymethyl)aminomethane) is an alkaline substance with a buffering capacity, capable of simultaneously performing pH adjustment and pH buffering.

Further, a pH value of the first liquid skin care composition is 4.5-7.0, and a pH value of the second liquid skin care composition is 6.0-8.0. In the present embodiment, the first liquid skin care composition maintains a slightly acidic to neutral pH, being beneficial for the stability and activity of skin conditioning ingredients (such as vitamin C derivatives, niacinamide, etc.). After the second liquid skin care composition is neutralized by a pH adjusting agent, a pH value is raised to neutral or slightly alkaline so that carbomer fully swells to form a transparent gel.

When the two liquid compositions are mixed, an acidic ingredient or a slightly acidic aqueous phase in the first liquid composition may undergo pH neutralization with an alkaline colloid system in the second liquid composition, causing an overall pH value to shift toward neutrality. At the same time, an aqueous phase and a polyol in the first liquid composition dilute a colloid concentration of the second liquid composition, but due to a high thickening efficiency of carbomer, even after dilution, a mixed system may maintain a gel-like texture. In addition, a pH change may induce further rearrangement of carbomer molecular chains, forming a new crosslinked network and enhancing viscoelasticity of a system.

In the present embodiment, by controlling a pH difference between the two liquid compositions, a rapid texture transformation during mixing is achieved. This transformation is primarily a physical process and does not involve a chemical reaction, thus having good stability and high repeatability.

It may be understood that, in some embodiments, pH values of the first liquid skin care composition and the second liquid skin care composition may be adjusted. As long as a pH difference between the two is within a reasonable range, a thickening effect after mixing may be achieved. For example, combinations such as a first liquid pH of 5.0 and a second liquid pH of 7.5; or a combination of a first liquid pH of 6.5 and a second liquid pH of 8.0 may all achieve similar technical effects

In one embodiment, a usage ratio of the first liquid skin care composition to the second liquid skin care composition is 1:10 to 10:1. This wide ratio range provides a user with great formulation flexibility.

When a ratio is 1:10 (first liquid:second liquid), a product is mainly the second liquid skin care composition, and after mixing, a high-viscosity gel texture is presented, with strong moisturizing and water-locking ability, suitable for dry skin or winter use. When a ratio is 10:1, a product is mainly the first liquid skin care composition, and after mixing, a relatively light texture is still maintained, with a high concentration of efficacy ingredients, suitable for oily skin or summer use.

Further, in one embodiment, a preferred usage ratio is 1:0.5 to 1:3 (first liquid:second liquid). Within this ratio range, a mixed product has both efficacy and comfortable skin feel, having both a sufficient concentration of efficacy ingredients and being capable of forming a moderate gel texture, providing a good moisturizing film-forming effect. The first liquid composition and the second liquid composition may be mixed in different ratios, and the structural transformation may occur across a range of mixing ratios.

Specifically, when a ratio is 1:0.5, a product tends to have a light texture, but a slight gel feel begins to appear; when a ratio is 1:1, the two liquids are mixed in equal amounts, obtaining a balanced texture and efficacy; when a ratio is 1:2 or 1:3, a gel feel is more obvious, and a moisturizing and water-locking ability is enhanced.

In the present embodiment, by providing a wide and adjustable usage ratio, a user may flexibly adjust a ratio according to factors such as own skin type, seasonal changes, and skin state, achieving personalized skin care. This usage method breaks the pattern of traditional skin care products that are fixed to a single bottle product and gives a user more autonomous choice. The first liquid skincare composition and the second liquid skincare composition may be combined in different ratios, and structural reorganization may occur across a range of mixing ratios.

It may be understood that, in some embodiments, a usage guide or a graduation mark may also be provided to help a user quickly find a suitable ratio. For example, hints such as “Refreshing Type 1:0.5”, “Balanced Type 1:1”, “Moisturizing Type 1:2” may be marked on a container to facilitate user selection.

In one embodiment, the contact mixing includes two methods. The contact mixing may occur prior to application, during application, or directly on the skin surface.

In a first method, after mixing the first liquid skin care composition and the second liquid skin care composition in a palm, a user applies a mixture to a skin surface. The user takes an appropriate amount of the first liquid skin care composition in a palm, then takes an appropriate amount of the second liquid skin care composition, and rubs and mixes the first liquid skin care composition and the second liquid skin care composition in a palm. During a mixing process, a texture may be felt gradually transforming from a liquid to a gel-like state. After sufficient mixing, the user applies a mixture to a facial skin. Advantages of this method may be that mixing is more sufficient and uniform, and the user may intuitively feel a texture change before application, facilitating control of a mixing degree. At the same time, a palm temperature may help to accelerate a mixing process and shorten a texture transformation time.

In a second method, after separately applying the first liquid skin care composition and the second liquid skin care composition to a skin surface, the user mixes the first liquid skin care composition and the second liquid skin care composition on the skin surface. The user first takes an appropriate amount of the first liquid skin care composition and applies the first liquid skin care composition to a face, then takes an appropriate amount of the second liquid skin care composition and applies the second liquid skin care composition to the face, and through tapping or massaging techniques, mixes the two compositions on the skin surface to form a gel-like texture.

Advantages of this method may be that zonal application may be achieved. For example, less of the second liquid may be used in a T-zone, and more of the second liquid may be used in a U-zone, performing differentiated care for different regional skin types. In addition, this method may allow a larger contact area between a product and skin, which may be beneficial for absorption of efficacy ingredients.

Additionally, the first liquid skin care composition and the second liquid skin care composition may be mixed in a container and then applied to skin.

In the present embodiment, both mixing methods may achieve a texture transformation, and a user may select according to personal habits and skin care needs. Regardless of the method, a mixing process occurs instantaneously during use, rather than pre-mixing during product production. The structural transformation described herein may occur before application, during application, or directly on the skin surface, and is not limited to any specific mixing location or sequence.

It may be understood that, in some embodiments, other mixing methods may also be used, such as mixing using a dedicated mixing tool (e.g., a spatula, a sponge, etc.), or mixing in a specific mixing container and then using a mixture. As long as sufficient contact between the two liquid compositions may be achieved, all such methods shall fall within the protection scope of the present disclosure.

In the present embodiment, the physical structure reorganization includes: an aqueous phase and a polyol system in the first liquid skin care composition interacting with a polymeric colloid network in the second liquid skin care composition, inducing physical rearrangement of a polymeric colloid structure. After the first liquid skin care composition and the second liquid skin care composition are contacted on a skin surface, physical structure reorganization may occur within 60 seconds and a viscoelastic gel-like structure may be formed, preferably within 10-30 seconds, significant thickening appears and a viscoelastic gel feel is formed; after spreading and standing on a skin surface, a film-forming state may be further exhibited within several minutes to several hours. The structural transformation described herein is not limited to a gel structure and may include various structured states such as viscoelastic structures, network structures, film-forming structures, or other structured phases generated during use.

Referring to FIG. 1 and FIG. 2, in the second liquid skin care composition, carbomer molecules have fully swelled under an action of a pH adjusting agent, forming a three-dimensional network structure. When the first liquid skin care composition is added, an aqueous phase and a polyol (e.g., glycerin, butylene glycol, etc.) therein may penetrate into a carbomer colloid network, leading to the following several physical changes.

First, a dilution effect may occur in that water in the first liquid may dilute a colloid concentration of the second liquid, but due to a high thickening efficiency of carbomer, even if a concentration decreases, a system may still maintain a relatively high viscosity.

Second, a solvation effect may occur in that polyol molecules may undergo hydrogen bonding interactions with carboxyl groups and hydroxyl groups on carbomer molecular chains, enhancing an extension degree of the molecular chains and improving viscoelasticity of a system.

Third, a pH adjustment effect may occur in that an acidic ingredient or a slightly acidic aqueous phase in the first liquid may undergo neutralization with an alkaline system in the second liquid, and a pH change may induce rearrangement of carbomer molecular chains, forming a new crosslinked network.

Fourth, an ionic strength effect may occur in that an electrolyte (e.g., a salt of an efficacy ingredient) in the first liquid may change an ionic strength of a system, affecting electrostatic repulsion of carbomer molecular chains and further regulating a network structure.

The above physical processes occur rapidly during a mixing process, typically completing within 30-60 seconds. A user may intuitively feel a texture transformation from a liquid to a gel-like state. This transformation is a reversible physical process. If further diluted with water, a viscosity decreases; if more of the second liquid is added, a viscosity further increases.

In the present embodiment, by designing ingredient compositions and pH values of the two liquid compositions, rapid and controllable physical structure reorganization is achieved. This instantaneous transformation mechanism gives a product a unique use experience while ensuring stability of efficacy ingredients. The structural reorganization described herein may occur through various physicochemical interactions and is not limited to any specific mechanism.

It may be understood that, in some embodiments, a transformation speed and a final texture may be controlled by adjusting parameters such as a carbomer content, a pH adjusting agent amount, a polyol content in the first liquid, etc. For example, increasing a carbomer content may shorten a transformation time and increase a final viscosity; increasing a polyol content may enhance a solvation effect and make a gel more stable.

In one embodiment, the skin conditioning ingredient includes at least one of: an antioxidant ingredient, a whitening ingredient, and a skin barrier repair ingredient, or a combination thereof. These efficacy ingredients give the first liquid skin care composition core skin care functions.

The antioxidant ingredient includes at least one of: a vitamin C derivative, carnosine, and Ectoin, or a combination thereof. In the present embodiment, 3-O-ethylascorbic acid (a vitamin C derivative) is preferably used, with a content of 1%-5%. This ingredient has excellent antioxidant and whitening effects and has better stability than ascorbic acid. Carnosine, as a natural anti-glycation and antioxidant ingredient, with a content of 0.1%-1%, may reduce advanced glycation end product formation and delay skin aging. Ectoin, as a natural protective ingredient, with a content of 0.5%-2%, may protect skin from environmental stress damage.

In the present embodiment, a whitening ingredient may include at least one of: niacinamide, kojic acid, and arbutin, with a content of 2%-5%, used to inhibit melanin production and improve skin tone. A skin barrier repair ingredient may include at least one of: ceramide, squalane, and cholesterol, used to repair a skin barrier and enhance moisturizing and water-locking ability.

By arranging these efficacy ingredients in the first liquid skin care composition and maintaining a slightly acidic to neutral pH environment, stability and activity of these ingredients may be maximally ensured. Degradation and inactivation problems that may be caused by long-term storage in an alkaline thickening system are avoided.

In the present embodiment, the first liquid skin care composition undertakes a main efficacy delivery function, providing the skin with multiple effects such as antioxidant, whitening, repair, through a high-concentration active ingredient formulation. When mixed with the second liquid skin care composition for use, these efficacy ingredients, wrapped in a gel texture, may better adhere to a skin surface, prolong an action time, and enhance efficacy performance.

It may be understood that, in some embodiments, other types of skin conditioning ingredients may also be added, such as anti-inflammatory ingredients (dipotassium glycyrrhizate, Centella asiatica extract), soothing ingredients (allantoin, panthenol), anti-aging ingredients (coenzyme Q10, hyaluronic acid), combined according to product positioning and target efficacy.

In one embodiment, the second liquid skin care composition further includes a moisturizing ingredient, used to enhance a moisturizing and water-locking ability of a gel after mixing.

The moisturizing ingredient includes at least one of: hyaluronic acid, sodium hyaluronate, and glycerin, or a combination thereof. In the present embodiment, hyaluronic acid, as a macromolecular moisturizing agent, with a content of 0.01%-0.1%, may form a moisturizing film on a skin surface and lock in moisture. Sodium hyaluronate, as a small-molecule moisturizing agent, with a content of 0.01%-0.2%, has a smaller molecular weight, may penetrate into a stratum corneum, and provide deep moisturizing. Glycerin, as a classic polyol moisturizing agent, with a content of 3%-10%, has both hygroscopic and water-locking effects.

In the present embodiment, by arranging a moisturizing ingredient in the second liquid skin care composition, the second liquid skin care composition has a moisturizing effect even when used alone, and a moisturizing effect is further enhanced when used in combination. A gel-like texture forms a continuous moisturizing film on a skin surface, reducing transepidermal water loss, while ingredients such as hyaluronic acid therein continuously replenish moisture to skin.

By arranging a thickening ingredient and a moisturizing ingredient in the second liquid, a design concept of structure as function is achieved. A gel network structure itself is a carrier for moisturizing and water-locking, without requiring an additional film former, simplifying a formulation and improving a skin feel.

In the present embodiment, the second liquid skin care composition undertakes a moisturizing film-forming function, complementing an efficacy delivery function of the first liquid skin care composition. When the first liquid skin care composition and the second liquid skin care composition are combined for use, a synergistic effect combining efficacy and moisturizing is achieved, having both efficacy performance of active ingredients, and moisturizing and water-locking of a gel texture, comprehensively improving a skin care effect.

It may be understood that, in some embodiments, other types of moisturizing ingredients, such as butylene glycol, 1,2-pentanediol, trehalose, and polyglutamic acid, or natural moisturizing factor (NMF) ingredients, such as urea, sodium lactate, and sodium PCA, may also be added to further enhance a moisturizing effect.

In the present disclosure, the skin care method of the present disclosure further includes a separate use mode: using the first liquid skin care composition alone, the first liquid skin care composition maintaining a liquid state on a skin surface; or using the second liquid skin care composition alone, the second liquid skin care composition maintaining a liquid or semi-flowable state on a skin surface.

When the first liquid skin care composition is used alone, a product exhibits a light watery texture, absorbs quickly, is non-greasy, suitable for oily skin or summer use, or as a pre-essence for subsequent skin care steps. At this time, a user obtains care with a high concentration of efficacy ingredients without an additional moisturizing burden.

When the second liquid skin care composition is used alone, a product exhibits a gel or light gel texture, has a certain moisturizing film-forming ability, suitable for use as a daily moisturizing product, or as an occlusive product after efficacy-type skin care products to help lock in active ingredients from previous steps.

In the present disclosure, by providing three use modes, first liquid alone, second liquid alone, and mixing of the two, a user is given great use flexibility. A user may select a most suitable use method according to factors such as skin state of the day, weather conditions, and subsequent skin care steps.

For example, in hot summer weather, a user may use the first liquid alone in the morning (refreshing effect) and mix in the evening (efficacy+moisturizing); in dry winter weather, mixing may be used both morning and evening, and a proportion of the second liquid may be increased; when a skin state is good, the first liquid alone may be used; when a skin barrier is damaged, the second liquid alone may be used or a proportion of the second liquid may be increased, focusing on moisturizing and repair.

In the present embodiment, by providing separate use modes, the skin care solution of the present disclosure is not limited to dual-liquid mixing, but covers a full set of use methods from a single liquid to dual-liquid mixing, maximizing practical value and cost performance of a product.

It may be understood that, in some embodiments, use suggestions or skin care plans may also be provided to guide a user to select an appropriate use mode in different scenarios, helping a user to fully utilize product efficacy.

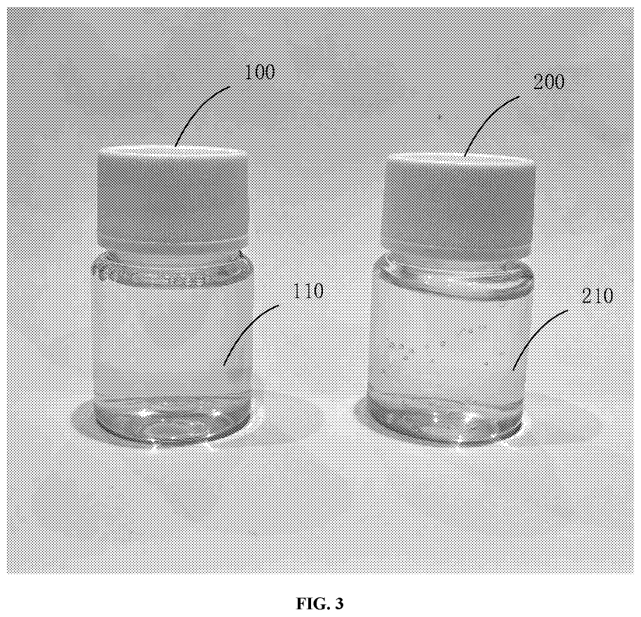

Referring to FIG. 3, the present disclosure further provides a dual liquid skin care set. The dual liquid skin care set includes a first container 100 and a second container 200. The first container 100 contains a first liquid skin care composition 110. The first liquid skin care composition 110 is a flowable liquid system and includes a skin conditioning ingredient. The second container 200 contains a second liquid skin care composition 210. The second liquid skin care composition 210 is a semi-flowable gel system and includes a pH adjusting ingredient. After the first liquid skin care composition 110 and the second liquid skin care composition 210 are contact-mixed during a skin use process, through physical structure reorganization, a system may be instantaneously transformed from a liquid or semi-flowable state into a viscoelastic gel-like structure or a film-forming state.

In one embodiment, the first container may adopt a packaging form such as a glass bottle, a plastic bottle, or a tube, with a capacity of 30-100 ml, equipped with a dispensing device such as a dropper, a pump, or a squeeze opening, for convenient user access.

In the present embodiment, the second container may adopt a packaging form such as a wide-mouth jar, a squeeze bottle, or a push bottle, with a capacity of 30-100 ml, for convenient user scooping or squeezing use.

In the present embodiment, the first container 100 and the second container 200 are in separate packaging, and both may be sold separately or as a combination. Advantages of separate packaging may be that a user may replenish separately according to usage amounts, avoiding waste; the two compositions may be stored separately, ensuring stability; and a user may carry one bottle alone for use, convenient for travel.

In the present embodiment, the dual liquid skin care set, as a product form, transforms the skin care method of the present disclosure into a marketable product, convenient for user purchase and use. A set may be accompanied by an instruction manual to guide a user on how to select a use mode, adjust a mixing ratio, customize a skin care plan according to skin type and season, etc.

It may be understood that, in some embodiments, the first container and the second container may be designed as conjoined packaging, such as a dual-chamber bottle, a dual-head bottle, etc., convenient for a user to simultaneously access the two compositions. A graduation mark may also be arranged on a container to help a user precisely control an amount and a ratio.

In the present embodiment, the first container and the second container are in separate packaging, and both are combined into a set product through an outer packaging box. A separate packaging design allows a user to flexibly select separate purchase or set purchase, meeting different needs.

A pH value of the first liquid skin care composition is in a range of 4.5-7.0, and a pH value of the second liquid skin care composition is in a range of 6.0-8.0. In the present embodiment, by controlling a pH difference between the two compositions, effective pH adjustment may occur during mixing, inducing physical structure reorganization. A slightly acidic environment of the first liquid ensures stability of efficacy ingredients, and a neutral to slightly alkaline environment of the second liquid allows a thickener to fully function.

In some embodiments, the polymeric thickener includes carbomer, and a content is 0.1%-2% of a total weight of the second liquid skin care composition; the pH adjusting ingredient includes triethanolamine.

Specific formulation examples are as follows. A composition of the first liquid skin care composition (in weight percentage) may include 70%-85% of deionized water, 5%-15% of a skin conditioning ingredient (such as niacinamide, a vitamin C derivative, Ectoin, and ceramide), 3%-8% of a moisturizer (such as glycerin and butylene glycol), 1%-5% of a plant extract, 0.2%-1% of a preservative, and an appropriate amount of other auxiliaries. A composition of the second liquid skin care composition (in weight percentage) may include 75%-90% of deionized water, 0.1%-2% (preferably 0.6%) of carbomer, 0.1%-2% (preferably 0.6%) of triethanolamine, 5%-15% of a moisturizing ingredient (such as glycerin, sodium hyaluronate, and panthenol), 0.05%-0.5% of xanthan gum, 0.2%-1% of a preservative, and an appropriate amount of other auxiliaries.

In the present embodiment, a molar ratio of carbomer to triethanolamine is preferably 1:1 to 1:1.2, ensuring sufficient neutralization of carbomer and formation of a stable transparent gel. When the first liquid and the second liquid are mixed at a ratio of 1:1, a carbomer concentration in a final formulation is 0.3% (if the second liquid contains 0.6% carbomer), and may still provide a sufficient thickening effect and form a gel texture.

After the first liquid skin care composition and the second liquid skin care composition are mixed on a skin surface, transformation to a gel-like structure or a film-forming state is completed within no more than 60 seconds. In the present embodiment, when a user mixes the two compositions in a palm or on a skin surface, an obvious texture change may typically be felt within 10-30 seconds, and a complete transformation to a stable gel state occurs within 30-60 seconds.

A user may intuitively feel a process of a liquid changing to a gel, enhancing fun and technological sense of a product. At the same time, rapid transformation also ensures use convenience, avoiding a long waiting time.

In the present embodiment, by precisely designing a formulation composition and ingredient proportions, instantaneous physical structure reorganization during use is achieved. This is a core innovative point distinguishing from prior art (pre-mixing preparation) and is also the most important technical contribution of the present disclosure.

It may be understood that, in some embodiments, a transformation speed and a final texture may be finely adjusted by adjusting parameters such as a carbomer content, a triethanolamine amount, a polyol content in the first liquid to adapt to different product positioning and target population needs.

The present disclosure is further elaborated below in conjunction with specific examples. It must be pointed out that examples are only for further illustrating the present disclosure and cannot be understood as limiting a protection scope of the present disclosure. Those skilled in the art may make some non-essential improvements and adjustments based on the content of the present disclosure described above. Test methods in the following examples without specifying specific conditions are usually performed under conventional conditions or under conditions suggested by manufacturers. Unless otherwise specified, all percentages and parts are by weight.

Specific formulation examples are as follows.

For Example 1 (Standard Formulation), 72.4 g of deionized water, 10.0 g of Hamamelis virginiana extract, 4.0 g of butylene glycol, 3.0 g of glycerin, 3.0 g of niacinamide, 3.0 g of 3-O-ethylascorbic acid, 1.2 g of cactus stem extract, 1.0 g of Ectoin, 3.33 g of ceramide NP (raw material with a content of 0.3%), 0.3 g of carnosine, 0.2 g of allantoin, 0.2 g of phenoxyethanol, 0.1 g of dipotassium glycyrrhizate, and 0.1 g of ethylhexylglycerin were sequentially added to a beaker and stirred at 200 rpm for 30 minutes using a stirring rod to uniformly disperse each ingredient, obtaining a first liquid skin care composition (essence water A) with a pH value of approximately 5.5-6.5. For preparing a second liquid skin care composition, 83.4 g of deionized water, 5.0 g of glycerin, 4.0 g of butylene glycol, 2.0 g of panthenol, 2.0 g of xylitol, 1.0 g of 1,2-pentanediol, 0.6 g of carbomer, 0.6 g of triethanolamine, 0.5 g of 1,2-hexanediol, 0.5 g of hydroxyacetophenone, 0.1 g of allantoin, 1.0 g of coenzyme Q10 (10% solution), 0.1 g of xanthan gum, 0.05 g of hyaluronic acid, and 0.05 g of sodium hyaluronate were sequentially added to a 250 ml beaker. The carbomer was first dispersed in a portion of water and stirred at 200 rpm for 10 minutes to fully disperse, then triethanolamine was added for neutralization and stirred for 30 minutes, and then other ingredients were sequentially added and stirring was continued for 30 minutes to uniformly mix each ingredient, obtaining a second liquid skin care composition (essence water B) with a pH value of approximately 6.5-7.5. Subsequently, the first liquid skin care composition and the second liquid skin care composition were mixed in a palm at a ratio of 1:1 to 1:2, forming a medium-viscosity gel texture within approximately 30 seconds.

For Example 2 (Low-Viscosity Gel Formulation), different from Example 1, in the second liquid skin care composition, carbomer was 0.1 g, triethanolamine was 0.12 g, deionized water was 84.2 g, and 0.38 g of trehalose was added, while other ingredients and a preparation method were the same. After mixing with the first liquid at a ratio of 1:1, a light gel texture with good flowability was formed within approximately 45 seconds, suitable for summer use and for oily skin.

For Example 3 (High-Viscosity Gel Formulation), different from Example 1, in the second liquid skin care composition, carbomer was 2.0 g, triethanolamine was 2.0 g, deionized water was 81.6 g, xanthan gum was 0.2 g. After dispersing carbomer in a portion of water, the mixture was stirred at 500 rpm for 15 minutes to fully swell, triethanolamine was added for neutralization and stirred for 40 minutes until a transparent gel was formed, and other operations were the same. After mixing with the first liquid at a ratio of 1:0.5 to 1:1, a thick gel texture with strong viscoelasticity was formed within approximately 15 seconds, suitable for winter use or for dry skin.

For Example 4 (Compounded Thickener System), different from Example 1, in the second liquid skin care composition, carbomer was 0.5 g, triethanolamine was 0.5 g, xanthan gum was 0.2 g, deionized water was 83.2 g, sodium hyaluronate was 0.1 g, trehalose was 0.25 g. Carbomer and xanthan gum were first compounded and dispersed in a portion of water, stirred at 300 rpm for 20 minutes, and then triethanolamine was added for neutralization, and other operations were the same. After mixing with the first liquid at a ratio of 1:1 to 1:2, a stable gel texture with good thixotropy and excellent skin feel was formed within approximately 25 seconds.

Table 1 shows key ingredient contents and performance parameters after mixing of the second liquid skin care compositions of Examples 1-4.

TABLE 1

| Key Parameter | Example 1 | Example 2 | Example 3 | Example 4 |
|---|---|---|---|---|
| Carbomer | 0.6 | 0.1 | 2 | 0.5 |
| Content (%) | | | | |
| Triethanolamine | 0.6 | 0.12 | 2 | 0.5 |
| Content (%) | | | | |
| Xanthan Gum | 0.1 | 0.1 | 0.2 | 0.2 |
| Content (%) | | | | |
| Recommended | 1:1 to 1:2 | 1:1 | 1:0.5 to 1:1 | 1:1 to 1:2 |
| Mixing Ratio | | | | |
| (A:B) | | | | |
| Gelation Time | approx. 30 | approx. 45 | approx. 15 | approx. 25 |
| (seconds) | | | | |
| Final Texture | Medium- | Light Gel | Thick Gel | Stable Gel |
| | Viscosity Gel | | | |
| Applicable Scene | All Seasons | Summer / | Winter / | All Skin |
| | | Oily Skin | Dry Skin | Types |

After mixing with the first liquid skin care composition (essence water A), all examples achieve instantaneous gelation within 15-45 seconds, satisfying a requirement of “no more than 60 seconds” defined in claim 10. As a carbomer content increases from 0.1% to 2.0%, a gelation time gradually shortens (45 seconds→30 seconds→15 seconds), and a final viscosity gradually increases, providing a technical basis for a user to select products with different textures according to needs.

With the above Examples 1-4, it is demonstrated that the technical solution of the present disclosure can achieve expected texture transformation effects under different thickener contents and different mixing ratios, with good adaptability and stability, fully supporting the technical solutions in the claims.

For Example 5 (Antioxidant Efficacy Formulation), 76.3 g of deionized water, 5.0 g of Hamamelis virginiana extract, 4.0 g of butylene glycol, 3.0 g of glycerin, 3.0 g of 3-O-ethylascorbic acid, 1.0 g of Ectoin, 0.5 g of carnosine, 0.5 g of alpha-lipoic acid, 0.1 g of astaxanthin, 1.0 g of coenzyme Q10 (10% solution), 0.2 g of allantoin, 0.3 g of phenoxyethanol, 0.1 g of ethylhexylglycerin, 0.5 g of vitamin E, and 0.1 g of dipotassium glycyrrhizate were sequentially added to a 250 ml beaker and stirred at 200 rpm for 30 minutes to uniformly disperse each ingredient, obtaining a first liquid skin care composition (antioxidant efficacy type) with a pH value in a range of approximately 5.5-6.5. This formulation is rich in multiple antioxidant ingredients, including a vitamin C derivative, carnosine, lipoic acid, astaxanthin, and coenzyme Q10, forming a powerful antioxidant system and being suitable for young mature skin and anti-aging needs.

For Example 6 (Whitening Efficacy Formulation), different from Example 5, deionized water was 75.9 g, and an antioxidant ingredient combination was replaced with 3.0 g of niacinamide, 2.0 g of 3-O-ethylascorbic acid, 1.0 g of kojic acid, 1.0 g of alpha-arbutin, 0.5 g of tranexamic acid, 1.0 g of Glycyrrhiza glabra root extract, 0.2 g of dipotassium glycyrrhizate, and 0.3 g of vitamin E, while other operations were the same, obtaining a whitening efficacy type formulation with a pH value in a range of approximately 5.0-6.0. This formulation may exert whitening effects through multiple pathways such as tyrosinase inhibition and melanin transport blockade, being suitable for pigmented and uneven skin tone.

For Example 7 (Barrier Repair Formulation), different from Example 5, deionized water was 75.6 g, Hamamelis virginiana extract was 3.0 g, and an antioxidant ingredient combination was replaced with 2.0 g of panthenol, 2.0 g of squalane, 3.33 g of ceramide NP, 1.67 g of ceramide AP, 1.67 g of ceramide EOP, 1.0 g of Centella asiatica extract, 0.5 g of hydroxyproline, 0.3 g of allantoin, 0.5 g of beta-glucan, and 0.2 g of cholesterol, while other operations were the same, obtaining a barrier repair type formulation with a pH value in a range of approximately 5.5-6.5. This formulation may mimic a skin lipid structure and comprehensively repair a barrier, being suitable for sensitive skin and barrier-damaged skin.

For Example 8 (Multi-Effect Composite Formulation), different from Example 5, deionized water was 73.8 g, and a single efficacy ingredient combination was replaced with composite ingredients including 3.0 g of niacinamide, 2.0 g of 3-O-ethylascorbic acid, 0.3 g of carnosine, 1.0 g of coenzyme Q10, 1.67 g of ceramide NP, 1.0 g of panthenol, 0.5 g of Centella asiatica extract, and 0.2 g of sodium hyaluronate, while other operations were the same, obtaining a multi-effect composite type formulation with a pH value in a range of approximately 5.5-6.5. This formulation integrates whitening, antioxidant, and repair ingredients, being suitable for mixed skin types and multiple skin care needs.

Table 2 shows a comparison of core efficacy ingredients of the first liquid skin care compositions of formulation Examples 5-8 (in the table, “-” indicates that the ingredient is not contained).

TABLE 2

| | Efficacy Ingredient Type | | | |
|---|---|---|---|---|
| | Example 5 | Example 6 | Example 7 | Example 8 |
| Efficacy | Antioxidant/ | Whitening/ | Barrier | Multi-Effect |
| Positioning | Anti-aging | Spot Fading | Repair | Composite |
| Niacinamide | — | 3 | — | 3 |
| (%) | | | | |
| 3-O- | 3 | 2 | — | 2 |
| Ethylascorbic | | | | |
| Acid (%) | | | | |
| Carnosine (%) | 0.5 | — | — | 0.3 |
| Ectoin (%) | 1 | — | 1 | 1 |
| Alpha-Lipoic | 0.5 | — | — | — |
| Acid (%) | | | | |
| Astaxanthin | 0.1 | — | — | — |
| (%) | | | | |
| Coenzyme | 0.1 | — | — | 0.1 |
| Q10 (%) | | | | |
| Kojic Acid (%) | — | 1 | — | — |
| Alpha- | — | 1 | — | — |
| Arbutin (%) | | | | |
| Tranexamic | — | 0.5 | — | — |
| Acid (%) | | | | |
| Ceramide NP | — | — | 1 | 0.5 |
| (%) | | | | |
| Ceramide AP | — | — | 0.5 | — |
| (%) | | | | |
| Ceramide EOP | — | — | 0.5 | — |
| (%) | | | | |
| Squalane (%) | — | — | 2 | — |
| Panthenol (%) | — | — | 2 | 1 |
| Centella | — | — | 1 | 0.5 |
| asiatica | | | | |
| Extract (%) | | | | |
| Beta-Glucan | — | — | 0.5 | — |
| (%) | | | | |
| Cholesterol (%) | — | — | 0.2 | — |
| Applicable | Young | Spots/ | Sensitive/ | Mixed/ |
| Population | Mature/ | Dullness | Barrier | Comprehensive |
| | Anti-aging | | Damaged | Needs |

Examples 5-8 prepare four first liquid skin care compositions with different efficacy positioning. Example 5 focuses on antioxidant, compounding 5 antioxidant ingredients (VC derivative, carnosine, lipoic acid, astaxanthin, coenzyme Q10), forming a comprehensive antioxidant system.

Example 6 focuses on whitening, compounding 5 types of whitening ingredients (niacinamide, VC derivative, kojic acid, arbutin, tranexamic acid), exerting whitening effects through multiple pathways such as tyrosinase inhibition and melanin transport blockade.

Example 7 focuses on skin barrier repair, compounding 3 types of ceramides (NP, AP, and EOP) as well as cholesterol and squalane to mimic a lipid structure of skin, combined with soothing ingredients such as panthenol, Centella asiatica, comprehensively repairing a skin barrier.

Example 8 is a multi-effect composite formulation, integrating whitening, antioxidant, and repair ingredients, suitable for users with needs for multiple effects.

The first liquid skin care compositions of all Examples 5-8 may be mixed with any of the second liquid skin care compositions in Examples 1~4 to achieve an instantaneous gelation effect. By adjusting a combination of efficacy ingredients in the first liquid, the dual liquid skin care method of the present disclosure may adapt to different product positioning and target populations, with extremely high flexibility and adaptability, fully reflecting the universality of the technical solution of the present disclosure.

For Test 1 (Instantaneous Gelation Time Test), the first liquid and the second liquid were mixed in a palm at a ratio of 1:1, and a time from contact to formation of a stable gel was recorded. Results showed that for Example 1 (low carbomer) it was approximately 45 seconds, for Example 2 (standard formulation) it was approximately 30 seconds, and for Example 3 (high carbomer) it was approximately 15 seconds. It was concluded that all formulations completed texture transformation within 60 seconds, meeting design requirements.

For Test 2 (Viscosity Change Test), viscosity values before and after mixing were measured using a viscometer. Results showed that a viscosity of the first liquid alone was approximately 861 cps, a viscosity of the second liquid alone was approximately 3320 cps, and a viscosity after 1:1 mixing was in a range of approximately 5000-8000 cps. It was concluded that a viscosity significantly increased after mixing, forming an obvious gel texture.

For Test 3 (Ratio Applicability Range Test), the first liquid and the second liquid were mixed at different ratios to observe whether a gel was formed. Results showed that a ratio range of 1:10 to 10:1 may all form a certain degree of gel texture, with a best effect within a range of 1:0.5 to 1:3. It was concluded that implementability of a wide ratio range in the claims was verified.

For Test 4 (Stability Test), a mixed gel was allowed to stand on a skin surface for 30 minutes to observe a texture change. Results showed that a gel texture remained stable with no obvious liquefaction or delamination. It was concluded that a gel structure after mixing was stable and capable of continuously exerting a moisturizing film-forming effect.

For Test 5 (Usage Feeling Evaluation), 30 volunteers were invited to try and evaluate usage feeling and preference. Results showed that 85% of volunteers believed that a design with an adjustable ratio was very practical, 90% of volunteers were interested in a texture transformation experience, 80% of volunteers expressed that they would adjust a ratio according to seasons, and 75% of volunteers believed that a moisturizing effect after mixing was superior to separate use. It was concluded that a usage method of the present disclosure was widely recognized by users and had high market acceptance.

Through the above technical effect verification, it is demonstrated that the technical solution of the present disclosure is feasible, has significant technical effects, and has good practical value and market prospects.

The above mechanism description is only used to illustrate the feasibility of the embodiments and does not constitute a sole limitation on the structure reorganization method or triggering conditions of the present disclosure. The structural transformation may result from various physicochemical interactions and is not limited to any specific mechanism.

The above description only describes embodiments of the present disclosure, and is not intended to limit the present disclosure; various modifications and changes can be made to the present disclosure. Any modifications, equivalent substitutions, and improvements made within the spirit and scope of the present disclosure are intended to be included within the scope of the present disclosure.

1. A skin care method, comprising:
providing a first liquid skin care composition, the first liquid skin care composition being a flowable liquid system, the first liquid skin care composition comprising a skin conditioning ingredient;
providing a second liquid skin care composition, the second liquid skin care composition being a semi-flowable gel system, the second liquid skin care composition comprising a pH adjusting ingredient; and
contact-mixing the first liquid skin care composition and the second liquid skin care composition during a skin use process;
wherein after the first liquid skin care composition and the second liquid skin care composition are contacted on a skin surface, through physical structure reorganization, a system is instantaneously transformed from a liquid or semi-flowable state into a viscoelastic gel-like structure or a film-forming state.

2. The skin care method of claim 1, wherein the second liquid skin care composition further comprises a polymeric thickener selected from the group consisting of: an acrylic polymer, and xanthan gum.

3. The skin care method of claim 2, wherein the acrylic polymer is selected from the group consisting of: carbomer, and crosslinked polyacrylic acid, and a content of the acrylic polymer is in a range of 0.1%-2% of a total weight of the second liquid skin care composition.

4. The skin care method of claim 1, wherein the pH adjusting ingredient comprises an alkaline substance.

5. The skin care method of claim 4, wherein the alkaline substance is selected from the group consisting of: triethanolamine, sodium hydroxide, potassium hydroxide, and tromethamine.

6. The skin care method of claim 1, wherein a pH value of the first liquid skin care composition is in a range of 4.5-7.0, and a pH value of the second liquid skin care composition is in a range of 6.0-8.0.

7. The skin care method of claim 1, wherein a usage ratio of the first liquid skin care composition to the second liquid skin care composition is in a range of 1:10 to 10:1.

8. The skin care method of claim 7, wherein the usage ratio of the first liquid skin care composition to the second liquid skin care composition is in a range of 1:0.5 to 1:3.

9. The skin care method of claim 1, wherein the contact-mixing comprises: after mixing the first liquid skin care composition and the second liquid skin care composition in a palm, applying to a skin surface; or after separately applying the first liquid skin care composition and the second liquid skin care composition to a skin surface, mixing on the skin surface.

10. The skin care method of claim 1, wherein the physical structure reorganization comprises: an aqueous phase and a polyol system in the first liquid skin care composition interacting with a polymeric colloid network in the second liquid skin care composition, inducing physical rearrangement of a polymeric colloid structure; after the first liquid skin care composition and the second liquid skin care composition are contacted on a skin surface, physical structure reorganization occurs within 60 seconds and a viscoelastic gel-like structure is formed; after spreading and standing on the skin surface, a film-forming state is further exhibited within several minutes to several hours.

11. The skin care method of claim 1, wherein the skin conditioning ingredient is selected from the group consisting of: an antioxidant ingredient, a whitening ingredient, and a skin barrier repair ingredient.

12. The skin care method of claim 11, wherein the antioxidant ingredient is selected from the group consisting of: a vitamin C derivative, carnosine, and Ectoin.

13. The skin care method of claim 1, wherein the second liquid skin care composition further comprises a moisturizing ingredient.

14. The skin care method of claim 13, wherein the moisturizing ingredient is selected from the group consisting of: hyaluronic acid, sodium hyaluronate, and glycerin.

15. The skin care method of claim 1, further comprising: using the first liquid skin care composition alone, the first liquid skin care composition maintaining a liquid state on a skin surface; or using the second liquid skin care composition alone, the second liquid skin care composition maintaining a liquid or semi-flowable state on the skin surface.

16. A dual liquid skin care set, comprising:
a first container, the first container containing a first liquid skin care composition, the first liquid skin care composition being a flowable liquid system, the first liquid skin care composition comprising a skin conditioning ingredient; and
a second container, the second container containing a second liquid skin care composition, the second liquid skin care composition being a semi-flowable gel system, the second liquid skin care composition comprising a pH adjusting ingredient;
wherein after the first liquid skin care composition and the second liquid skin care composition are contact-mixed during a skin use process, through physical structure reorganization, a system is instantaneously transformed from a liquid or semi-flowable state into a viscoelastic gel-like structure or a film-forming state.

17. The dual liquid skin care set of claim 16, wherein the first container and the second container are in separate packaging.

18. The dual liquid skin care set of claim 16, wherein a pH value of the first liquid skin care composition is in a range of 4.5-7.0, and a pH value of the second liquid skin care composition is in a range of 6.0-8.0.

19. The dual liquid skin care set of claim 16, wherein the second liquid skin care composition comprises a polymeric thickener comprising carbomer, a content of the carbomer being in a range of 0.1%-2% of a total weight of the second liquid skin care composition; and the pH adjusting ingredient comprises triethanolamine.

20. The dual liquid skin care set of claim 16, wherein after the first liquid skin care composition and the second liquid skin care composition are mixed on a skin surface, transformation to a gel-like structure or a film-forming state is completed within no more than 60 seconds.
